(19) United States
(12) Patent Application Publication
(10) Pub. No.: US 2016/0067171 A1
(43) Pub. Date: Mar. 10, 2016

(54) ORAL AND DENTAL HYGIENE AND CLEANING AGENTS WITH OPTIMIZED TASTE

(71) Applicant: Henkel AG & Co. KGaA, Duesseldorf (DE)

(72) Inventors: Thomas Welss, Duesseldorf (DE); Thomas Foerster, Duesseldorf (DE); Markus Semrau, Langenfeld (DE); Kristin Miehlich, Wuppertal (DE); Claudia Hundeiker, Meerbusch (DE)

(21) Appl. No.: 14/944,983

(22) Filed: Nov. 18, 2015

Related U.S. Application Data
(63) Continuation of application No. PCT/EP2014/052035, filed on Feb. 3, 2014.

(30) Foreign Application Priority Data
May 28, 2013 (DE) 10 2013 209 893.6

(51) Int. Cl. A61K 8/85; A61K 8/24; A61K 8/21; A61Q 11/00; A61K 8/34

(52) U.S. Cl. CPC A61K 8/85; A61Q 11/00; A61K 8/345; A61K 8/21; A61K 8/24; A61K 2800/412; A61K 2800/49; A61K 2800/28; A61K 2800/596; A61K 2800/92

(57) ABSTRACT

The invention relates to oral and dental hygiene- and cleaning agents having an improved taste and a “silky sensation” in the mouth, said agents containing, in relation to their weight, between 0.001 and 25 wt. % poly(lactic acid) particles, between 0 and 15 wt. % sorbitol and between 0 and 30 wt. % glycerol, with the proviso that the total quantity of ingredient(s) in groups b) and c) lies between 5 and 40 wt. %.

### FIELD OF THE INVENTION

The present invention generally relates to oral and dental hygiene and cleaning agents which have been improved in terms of taste and which moreover take into account the particular requirements when using electric toothbrushes.

### BACKGROUND OF THE INVENTION

Dental cleaning agents are on the market in various forms and serve primarily for cleaning the tooth surface and for preventing dental and gum diseases. They usually contain a combination of polishing agents, humectants, surfactants, binders, flavorings and fluoride-containing and antimicrobial active ingredients. Besides tooth powders, which play a lesser role due to the increased abrasiveness thereof, dental cleaning agents are primarily provided in the form of paste, cream and translucent or transparent gel. In recent years, liquid tooth creams and mouthwashes have also increasingly gained importance.

In most cases, oral and dental hygiene and cleaning agents contain humectants from the polyol group. Sorbitol aside, in particular glycerol, xylitol and mannitol have proven useful, but humectants from other groups, for example polyethylene glycols, are also readily used.

One problem that occurs particularly when using sorbitol and glycerol is that the compositions are perceived by consumers as not having an optimal taste. In customer satisfaction surveys, a “sweet and soapy” or “oily” to “musty” taste and an unpleasant feeling in the mouth are described time and time again.

Another area of focus lies in providing products which solve their prescribed task with as little outlay on raw materials as possible, in order to be able to save resources and to provide products that are ecologically friendly. Reducing the amount of synthetic ingredients is therefore another important aim when formulating cosmetic agents.

The use of polylactic acid particles in oral hygiene products is described in international patent applications WO2012/177616 and WO2012/177617. In said documents, however, the particles are disclosed only as biodegradable abrasive substances. Details regarding the taste cannot be found therein, and moreover humectants are always used in large quantities in the exemplary compositions; in particular, the sorbitol content is at least 17%.

The object of the present invention was therefore to provide an oral and dental hygiene and cleaning agent which solves the aforementioned problems and which cannot be criticized in terms of taste. The intention was also for the formulations to contain the smallest possible quantity of synthetic ingredients.

It has now been found that the use of polylactic acid particles in compositions having a defined sorbitol and glycerol content leads to a considerably improved taste experience, and moreover an optimal product consistency is achieved which is deemed by consumers to have a “melting feeling in the mouth.” Moreover, these compositions are particularly suitable for cleaning using electric toothbrushes, since they are kind to the sensitive mechanics of the electrically operated brush heads.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An oral and dental hygiene and cleaning agent, containing—relative to its weight—a) 0.001 to 25% by weight polylactic acid particles, b) 0 to 15% by weight sorbitol, and c) 0 to 30% by weight glycerol, with the proviso that the total amount of ingredient(s) from groups b) and c) is 5 to 40% by weight.

The use of polylactic acid particles to improve the taste of oral and dental hygiene and cleaning agents which contain sorbitol and/or glycerol.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the present invention is, in a first embodiment, an oral and dental hygiene and cleaning agent, containing—relative to its weight—
a) 0.001 to 25% by weight polylactic acid particles, and
b) 0 to 15% by weight sorbitol,
c) 0 to 30% by weight glycerol,
   with the proviso that the total amount of ingredient(s) from groups b) and c) is 5 to 40% by weight.

Oral and dental hygiene agents and oral and dental cleaning agents in the context of the invention are oral and dental powders, oral and dental pastes, liquid oral and dental creams, oral and dental rinses and oral and dental gels. Toothpastes and liquid tooth cleaning agents are particularly suitable. To this end, the oral and dental hygiene and cleaning agents may be, for example, in the form of toothpastes, liquid tooth creams, tooth powders, mouthwashes or may optionally also exist as a chewable base, for example as a chewing gum. Preferably, however, they are in the form of more or less viscous or plastic toothpastes, as used for cleaning the teeth using a toothbrush. Another particularly preferred embodiment of the present invention lies in mouth rinsing solutions and mouthwashes, which are used for rinsing out the oral cavity.

As a first essential ingredient, the compositions according to the invention contain, relative to their weight, 0.001 to 25% by weight polylactic acid particles.

Agents which are preferred according to the invention use the polylactic acid particles within relatively narrow quantity ranges. Preference is given here to oral and dental hygiene and cleaning agents according to the invention which contain,—relative to their weight—0.002-20% by weight, preferably 0.003-17.5% by weight, particularly preferably 0.004-15% by weight, extremely preferably 0.005-12.5% by weight and in particular 0.01 to 10% by weight polylactic acid particles.

Polylactic acid, also known as polylactide or PLA, is a name given to biodegradable polymers (polyesters) which are obtainable in particular by the ionic polymerization of lactide, a ring-shaped association of two lactic acid molecules.

At temperatures between 140 and 180° C. and under the effect of catalytic tin compounds (for example tin oxide), a ring opening polymerization takes place. Synthetic substances of high molecular weight and strength are thus produced. Lactide itself can be produced by fermentation of molasses or by fermentation of glucose with the aid of various bacteria.

Pure polylactides of high molecular weight can also be produced by means of so-called polycondensation directly from lactic acid. In industrial production, however, the disposal of the solvent is problematic.

Lactic acid (2-hydroxypropanoic acid) has an asymmetric carbon atom, so that even polylactic acid has optically active centers in the L(+) and D(−) configuration. The ratio of L to D monomer units determines the degree of crystallinity, the melting point and the biodegradability of the polymers.

Polylactic acids which are suitable according to the invention are L-polylactic acid, D-polylactic acid and L/D-polylactic acid and mixtures thereof. On account of its very good biodegradability, L-polylactic acid is particularly preferred. In one preferred embodiment of the present invention, the weight proportion of L-lactic acid monomer units in the polylactic acid is greater than 50% by weight, preferably greater than 80% by weight and in particular greater than 90% by weight.

The molar mass of the polylactic acid is usually 1000 to 1,000,000, preferably 10,000 to 300,000, more preferably 50,000 to 250,000 and in particular 100,000 to 180,000 Dalton.

In a further preferred embodiment of the present invention, the polylactic acid is used in a form blended with fillers. The use of relatively large quantities of filler helps to comminute the polymer into particles and increases the biodegradability and the inner specific surface area via porosity and capillarity. Particular preference is given here to water-soluble fillers, for example metal chlorides such as NaCl, KCl, etc., metal carbonates such as Na₂CO₃, NaHCO₃, etc., metal sulfates such as MgSO₄.

As fillers, use may also be made of natural raw materials, for example nutshells, wood or bamboo fibers, starch, xanthan, alginates, dextran, agar, etc. These fillers are biodegradable and do not impair the good ecological properties of the polylactic acid particles.

The polylactic particle content in biodegradable fillers may usually be 10 to 70% by weight, preference being given to quantities of 20 to 60% by weight and particular preference being given to quantities of 30 to 50% by weight.

Although the polylactic acid particles in the context of the present invention are not used for their abrasive properties, irregular shapes have proven to be particularly preferred since the effect according to the invention can thus be further increased in comparison to spherical particles.

Particles which are particularly suitable according to the invention have a circularity of between 0.1 and 0.6.

The shape of the polylactic acid particles used according to the invention may be defined in various ways, wherein in the context of this preferred embodiment of the present invention the geometric proportions of a particle and—more practically—of a particle population are determined.

Highly precise modern methods enable the exact determination of particle shapes from a large number of particles, usually of more than 10,000 particles, preferably of more than 100,000 particles. These methods make it possible to select with precision the average particle shape of a particle population. Preferably, the particle shapes are determined using an “Occhio Nano 500 Particle Characterisation Instrument” using the software “Callistro Version 25” (Occhio s.a. Liege, Belgium). This device enables the preparation, dispersing, imaging and analysis of a particle population, the device parameters preferably being set as follows: white requested=180, vacuum time=5000 ms, sedimentation time=5000 ms, automatic threshold, number of particles counted/analyses=8000 to 500,000, minimum number of replicates/sample=3, lens setting 1×/1.5×.

The polylactic acid particles used according to the invention preferably have sizes which are characterized by their area-equivalent diameter (ISO 9276-6:2008(E) section 7), also called “Equivalent Circle Diameter ECD” (ASTM F1877-05 Section 11.3.2). The mean ECD of a particle population is calculated as the average ECD of each individual particle of a particle population of at least 10,000 particles, preferably more than 50,000 particles, in particular more than 100,000 particles, once particles having an equivalent circle diameter (ECD) of less than 10 μm have been excluded from the measurement.

In one preferred embodiment of the present invention, the polylactic acid particles have mean ECD values of 10 to 1000 μm, preferably 50 to 500 μm, more preferably 100 to 350 μm and in particular 150 to 250 μm.

Regardless of the mean particle size, preference is given to oral and dental hygiene and cleaning agents according to the invention in which the polylactic acid particles have absolute particle sizes of 1 to 1000 μm, preferably 2 to 750 μm and in particular 10 to 500 μm.

In the context of the present invention, use is made of shape descriptors which are calculations of geometric descriptors or shape factors. Shape factors are ratios between two different geometric properties, which are in turn a measurement of the proportions of the image of a whole particle or the measurement of the proportions of an ideal geometric body enveloping the particle.

These results are descriptors which are similar to aspect ratios. In one preferred embodiment of the present invention, meso-form descriptors are used for particle characterization. These meso-form descriptors indicate the extent to which a particle differs from an ideal geometric shape, in particular from a sphere. In the preferred embodiment of the present invention, the polylactic acid particles are different from the typical spherical shape or spherical-like shape, such as granulate particles for example.

Here, the particles preferably have sharp corners and edges and preferably have concave indentations. Sharp corners of non-spherical particles may be defined by a radius of less than 20 μm, preferably less than 8 μm and in particular less than 5 μm, the radius being defined as the radius of an imaginary circle following the contour of the corner.

Circularity is a quantitative, 2-dimensional image analysis and can be determined in accordance with ISO 9276-6:2008(E) section 8.2. Circularity is a preferred meso-form descriptor and can be determined for example using the above-described “Occhio Nano 500 Particle Characterisation Instrument” using the software “Callistro version 25” (Occhio s.a. Liege, Belgium) or using the “Malvern Morphologi G3.” Circularity is sometimes described in the literature as the difference between a particle and the perfect spherical shape. The values for circularity vary between 0 and 1, wherein 1 describes the perfect sphere or (in the two-dimensional image) the perfect circle:

C=[(4πA)/P²]^{1/2}

in which A is the projection area (the two-dimensional descriptor) and P is the length of the perimeter of the particle.

Polylactic acid particles having a mean circularity C of 0.1 to 0.6, preferably 0.15 to 0.4 and in particular 0.2 to 0.35 have proven to be particularly suitable in the context of the present invention. Here, the mean values are obtained by quotient formation from volume-based measurements and number-based measurements.

Solidity is a quantitative, 2-dimensional image analysis and can be determined in accordance with ISO 9276-6:2008(E) section 8.2. Solidity is also a preferred meso-form descriptor and can be determined for example using the above-described “Occhio Nano 500 Particle Characterisation Instrument” using the software “Callistro version 25” (Occhio s.a. Liege, Belgium) or using the “Malvern Morphologi G3.” Solidity is a meso-form descriptor which describes the concavity of a particle or of a particle population. The values for solidity vary between 0 and 1, wherein 1 describes a non-concave particle:

solidity=A/Ac

in which A is the (image) area of the particle and Ac is the area of the convex shell enclosing the particle.

Polylactic acid particles having a mean solidity of 0.4 to 0.9, preferably 0.5 to 0.8 and in particular 0.55 to 0.65 have proven to be particularly suitable in the context of the present invention. Here, the mean values are obtained by quotient formation from volume-based measurements and number-based measurements.

In particularly preferred embodiments of the present invention, the polylactic acid particles used have a mean circularity C of 0.1 to 0.6, preferably 0.15 to 0.4 and in particular 0.2 to 0.35 and a mean solidity of 0.4 to 0.9, preferably 0.5 to 0.8 and in particular 0.55 to 0.65.

The “mean” circularities and solidities are mean values from the measurement of a large number of particles, usually of more than 10,000 particles, preferably more than 50,000 particles and in particular more than 100,000 particles, wherein particles having an equivalent circle diameter (ECD) of less than 10 μm have been excluded from the measurement.

After being produced, the polylactic acid polymer can be converted into the desired particle size and shape, for example by means of grinding methods.

One particularly preferred method for producing the particles of desired circularity and solidity lies in producing a foam of polylactic acid and then grinding.

Although the polylactic acid particles in the context of the present invention are not used for their abrasive properties, polylactic acid particles of specific hardness have proven to be particularly preferred since the effect according to the invention can thus be further increased in comparison to particles that are too hard or too soft.

The hardness of the particles can be varied via the ratio of D to L monomers and via the molar mass.

Preferred polylactic acid particles have hardnesses of 3 to 50 kg/mm², preferably 4 to 25 kg/mm², and in particular 5 to 15 kg/mm² on the HV Vickers hardness scale.

As second essential ingredients, the agents according to the invention contain sorbitol and/or glycerol in a total quantity of 5 to 40% by weight. Compositions which contain only sorbitol accordingly contain 5 to 15% by weight sorbitol (lower limit from the total quantity condition, upper limit from the sorbitol quantity), compositions which contain only glycerol accordingly contain 5 to 30% by weight glycerol (lower limit from the total quantity condition, upper limit from the glycerol quantity), and compositions which contain both ingredients contain the mixture in a proportion of 5 to 40% by weight, the quantities of the individual ingredients being at most 15% by weight (sorbitol) and at most 30% by weight (glycerol).

In the context of the present application, all the quantities specified for sorbitol and/or glycerol relate to 100% strength active substances, even if other delivery forms (for example 70% strength sorbitol or 86% strength glycerol) might be used.

Oral and dental hygiene and cleaning agents which are preferred according to the invention are characterized in that the total amount of sorbitol b) and glycerol c) is 5 to 39% by weight, preferably 7.5 to 38% by weight, more preferably 10 to 37% by weight, even more preferably 15 to 36% by weight and in particular 20 to 35% by weight.

In one preferred embodiment, the compositions according to the invention contain sorbitol. Particularly preferred oral and dental hygiene and cleaning agents according to the invention are characterized in that they contain 1 to 14.5% by weight, preferably 2.5 to 14% by weight, more preferably 5 to 13.5% by weight and in particular 10 to 13% by weight sorbitol.

While in the first two quantity ranges mentioned glycerol can be contained in the compositions (in order to satisfy the total quantity condition), the compositions which have sorbitol contents within the two last-mentioned quantity ranges may also be free of glycero 1.

In one preferred embodiment, the compositions according to the invention contain glycerol. Particularly preferred oral and dental hygiene and cleaning agents according to the invention are characterized in that they contain 5 to 28% by weight, preferably 7.5 to 26% by weight, more preferably 10 to 25% by weight and in particular 12.5 to 24% by weight glycerol.

Since all the aforementioned quantity ranges for glycerol satisfy the total quantity condition, the preferred compositions mentioned above may be free of sorbitol. However, an additional sorbitol content may also be present within the limits defined under the condition.

The compositions according to the invention are preferably water-based. Particularly preferred oral and dental hygiene and cleaning agents according to the invention contain 40 to 84% by weight, preferably 45 to 80% by weight, more preferably 50 to 75% by weight, even more preferably 55 to 70% by weight and in particular 57.5 to 65% by weight water.

The choice of narrow quantity ranges for glycerol and sorbitol in combination with the polylactic acid particles leads to a considerable improvement in the taste experience. Even in comparison to compositions which contain the aforementioned quantities of glycerol and sorbitol but which are free of polylactic acid, the compositions according to the invention exhibit a considerably improved taste experience and feeling in the mouth.

Oral and dental hygiene and cleaning agents may particularly preferably also contain anti-cavity active ingredients. These may be selected for example from organic or inorganic fluorides, for example from sodium fluoride, potassium fluoride, sodium monofluorophosphate and sodium fluorosilicate. Zinc fluoride and tin(II) fluoride are also preferred. The compositions should preferably contain a quantity of 0.01-0.2% by weight fluorine in the form of the aforementioned compounds.

According to the invention, preference is given to oral and dental hygiene and cleaning agents according to the invention which additionally contain anti-cavity active ingredients, preferably fluorine compound(s), in particular sodium fluoride, potassium fluoride, sodium monofluorophosphate, zinc fluoride, tin fluoride and sodium fluorosilicate, preferably in quantities of 0.01 to 5% by weight, preferably in quantities of 0.01 to 5% by weight, preferably 0.02 to 2.5% by weight and in particular 0.04 to 1.1% by weight, in each case relative to the agent as a whole.

It has surprisingly been found that the fluoride deposition can be increased if polylactic acid particles are additionally contained in the compositions above certain fluoride contents. The minimum quantity of fluoride is in this case 1200 ppm; below this limit, the use of polylactic acid would have no noticeable effect on the fluoride deposition.

Oral and dental hygiene and cleaning agents which are preferred according to the invention are characterized in that they contain 1225 to 1575 ppm, preferably 1250 to 1550 ppm, more preferably 1275 to 1525 ppm, even more preferably 1300 to 1500 ppm, even more preferably 1325 to 1475 ppm and in particular 1350 to 1450 ppm fluoride.

If fluoride is provided in the form of sodium fluoride, 1% by weight sodium fluoride corresponds to approximately 4524 ppm fluoride, so that preferred agents according to the invention contain 0.27 to 0.35% by weight, preferably 0.28 to 0.34% by weight, more preferably 0.29 to 0.33% by weight and in particular 0.30 to 0.32% by weight sodium fluoride.

The oral and dental hygiene agents, in particular the toothpastes, may also contain substances which increase the insensitivity of the teeth, for example potassium salts such as, for example, potassium nitrate, potassium citrate, potassium chloride, potassium bicarbonate and potassium oxalate. Oral and dental hygiene and cleaning agents which are preferred according to the invention are characterized in that they contain substances which increase the insensitivity of the teeth, preferably potassium salts, particularly preferably potassium nitrate and/or potassium citrate and/or potassium chloride and/or potassium bicarbonate and/or potassium oxalate, preferably in quantities of 0.5 to 20% by weight, particularly preferably 1.0 to 15% by weight, more preferably 1.5 to 5% by weight and in particular 1.75 to 2.5% by weight, in each case relative to the agent as a whole.

The agents according to the invention may also additionally contain further wound-healing and anti-inflammatory substances, for example active ingredients against gum inflammation. Such substances may be selected for example from allantoin, azulene, chamomile extracts, tocopherol, panthenol, bisabolol, sage extracts.

The use of abrasives is likewise preferred. Abrasives are amorphous, predominantly inorganic, largely water-insoluble, fine powders which have no sharp edges. In dental and oral hygiene agents, they help to clean the teeth and at the same time polish the tooth surface (polishing agents).

Suitable polishing agents are in principle all friction bodies known for toothpastes, particularly those which contain no calcium ions. Polishing agent components which are preferably suitable are therefore silicas, aluminum hydroxide, aluminum oxide, sodium aluminum silicates, organic polymers or mixtures of such friction bodies.

Calcium-containing polishing components, such as, for example, chalk, calcium pyrophosphate, dicalcium phosphate dihydrate, may nevertheless be contained in quantities of up to 5% by weight, relative to the composition as a whole.

The total content of polishing agents preferably lies in the range from 5 to 50% by weight of the dental hygiene agent.

Particular preference is given to toothpastes and liquid tooth cleaning agents which contain silicas as polishing agents. Suitable silicas are, for example, silica gels, silica hydrogels and precipitated silicas. Silica gels are produced by reacting sodium silicate solutions with strong, aqueous mineral acids to form a hydrosol, aging to form the hydrogel, washing and drying. If the drying takes place under gentle conditions until water contents of 15 to 35% by weight are reached, the so-called silica hydrogels are obtained. By drying to water contents below 15% by weight, an irreversible shrinkage of the previously loose structure of the hydrogel to the dense structure of the so-called xerogel takes place.

The precipitated silicas are a second group of silica polishing agents, this group being suitable with preference. These are obtained by precipitation of silica from dilute alkali silicate solutions by adding strong acids under conditions at which the aggregation to the sol and gel cannot occur. Suitable methods for Particularly suitable is a precipitated silica having a BET surface area of 15-110 m²/g, a particle size of 0.5-20 μm, wherein at least 80% by weight of the primary particles should be less than 5 μm, and a viscosity in a 30% glycerol/water (1:1) dispersion of 30-60 Pa·s (20° C.) in a quantity of 10-20% by weight of the toothpaste. Precipitated silicas of this type which are preferably suitable also have rounded corners and edges and are available under the trade name Sident® 12 DS (DEGUS SA).

Other precipitated silicas of this type are Sident® 8 (DEGUSSA) and Sorbosil® AC 39 (Crosfield Chemicals). These silicas are characterized by a lesser thickening effect and a somewhat larger mean particle size of 8-14 μm with a specific surface area of 40-75 m²/g (according to BET) and are particularly suitable for liquid tooth creams. These should have a viscosity (25° C., shear rate D=10 s⁻¹) of 10-100 Pa·s.

Toothpastes, which have a much higher viscosity of more than 100 Pa·s (25° C., D=10 s⁻¹), in contrast require a sufficiently high proportion of silicas having a particle size of less than 5 μm, preferably at least 3% by weight of a silica having a particle size of 1-3 μm. There is thus added to such toothpastes, besides the aforementioned precipitated silicas, preferably even finer, so-called thickening silicas having a BET surface area of 150-250 m²/g, for example the commercially available products Sipernat 22 LS or Sipernat® 320 DS.

As further polishing agent components, the composition may also contain for example aluminum oxide in the form of weakly calcined alumina having a content of—and—aluminum oxide in a quantity of approximately 1-5% by weight. Such a suitable aluminum oxide is available under the trade name “Poliertonerde P10 feinst” (Giulini Chemie).

Also suitable as polishing agents are all friction bodies known for toothpastes, such as, for example, sodium aluminum silicates such as zeolite A for example, organic polymers such as polymethacrylate for example, or mixtures of these and of the aforementioned friction bodies.

To sum up, preference is given to oral and dental hygiene and cleaning agents according to the invention which additionally contain abrasives, preferably silicas, aluminum hydroxide, aluminum oxide, calcium pyrophosphate, chalk, dicalcium phosphate dihydrate (CaHPO₄.2H₂O), sodium aluminum silicates, in particular zeolite A, organic polymers, in particular polymethacrylates, or mixtures of these friction bodies, preferably in quantities of 1 to 30% by weight, preferably 2.5 to 25% by weight and in particular 5 to 22% by weight, in each case relative to the agent as a whole.

Particularly preferred oral and dental hygiene and cleaning agents according to the invention contain—relative to their weight—5 to 25% by weight, preferably 7.5 to 22.5% by weight, more preferably 10 to 20% by weight and in particular 12.5 to 18% by weight polishing agents from the group consisting of silicas, aluminum hydroxide, aluminum oxide, dicalcium phosphate dihydrate (CaHPO₄.2H₂O) or mixtures of these friction bodies.

The stated quantities relate to the total quantity of friction bodies, wherein individual friction bodies are preferably used in narrower quantity ranges. Agents which are preferred according to the invention contain for example 5 to 20% by weight, preferably 8 to 21% by weight, more preferably 9 to 20% by weight and in particular 11 to 19% by weight silica(s). Agents according to the invention which are more preferred are characterized in that they contain 0.25 to 2% by weight, preferably 0.5 to 1.5% by weight and in particular 0.75 to 1.25% by weight aluminum oxide.

To sum up, preference is given to oral and dental hygiene and cleaning agents according to the invention which additionally contain abrasives, preferably silicas, aluminum hydroxide, aluminum oxide, calcium pyrophosphate, chalk, dicalcium phosphate dihydrate (CaHPO₄.2H₂O), sodium aluminum silicates, in particular zeolite A, organic polymers, in particular polymethacrylates, or mixtures of these friction bodies, preferably in quantities of 1 to 30% by weight, preferably 2.5 to 25% by weight and in particular 5 to 22% by weight, in each case relative to the agent as a whole.

Oral and dental hygiene and cleaning agents may for example also contain substances which are effective against plaque and/or tartar.

Substances which are effective against tartar may for example be chelating agents such as, for example, ethylenediaminetetraacetic acid and its sodium salts, pyrophosphate salts such as the water-soluble dialkali or tetraalkali metal pyrophosphate salts, for example Na₄P₂O₇, K₄P₂O₇, Na₂K₂P₂O₇, Na₂H₂P₂O₇ and K₂H₂P₂O₇ or polyphosphate salts, which may be selected for example from water-soluble alkali metal tripolyphosphates such as sodium tripolyphosphate and potassium tripolyphosphate.

Oral and dental hygiene and cleaning agents which are preferred according to the invention are characterized in that they additionally contain phosphate(s), preferably alkali metal phosphate(s) and in particular sodium tripolyphosphate, preferably in quantities of 1 to 10% by weight, particularly preferably 2 to 8% by weight and in particular 3 to 7% by weight, in each case relative to the agent as a whole.

As consistency regulators (or binders), use is made, for example, of natural and/or synthetic water-soluble polymers such as alginates, carraghenates, tragacanth, starch and starch ethers, cellulose ethers such as, for example, carboxymethylcellulose (sodium salt), hydroxyethylcellulose, methylhydroxypropylcellulose, guar, acacia gum, agar agar, xanthan gum, succinoglycan gum, carob flour, pectins, water-soluble carboxyvinyl polymers (for example Carbopol® types), polyvinyl alcohol, polyvinylpyrrolidone, polyethylene glycols, particularly those having molecular weights of 1500-1,000,000.

Further substances suitable for controlling the viscosity are, for example, sheet silicates such as for example montmorillonite clays, colloidal thickening silicas, such as for example aerogel silicas, pyrogenic silicas or finely ground precipitated silicas. Viscosity-stabilizing additives from the group consisting of cationic, zwitterionic or ampholytic nitrogen-containing surfactants, hydroxypropyl-substituted hydrocolloids or polyethylene glycol/polypropylene glycol copolymers having a mean molar weight of 1000 to 5000 or a combination of the aforementioned compounds, may also be used in the toothpastes.

Surface-active substances can also be used in the agents according to the invention. In toothpastes, for example, these aid the cleaning effect and if desired also help to develop foam when brushing the teeth or when rinsing the mouth and help to stabilize the dispersion of the polishing bodies in the carrier and are used both in mouth rinsing solutions and in toothpastes usually in a quantity of 0.1 to 5% by weight.

Suitable surfactants are, for example, linear sodium alkyl sulfates having 12-18 carbon atoms in the alkyl group. These substances additionally have an enzyme-inhibiting effect on the bacterial metabolism of the plaque. Further suitable surfactants are alkali salts, preferably sodium salts of alkyl polyglycol ether sulfate having 12-16 carbon atoms in the linear alkyl group and 2-6 glycol ether groups in the molecule, of linear alkane (C₁₂-C₁₈) sulfonate, of sulfosuccinic acid monoalkyl (C₁₂-C₁₈) esters, of sulfated fatty acid monoglycerides, sulfated fatty acid alkanolamides, sulfoacetic acid alkyl (C₁₂-C₁₆) esters, acyl sarcosines, acyl taurides and acyl isothionates having in each case 8-18 carbon atoms in the acyl group. Zwitterionic, ampholytic and nonionic surfactants are also suitable, for example oxethylates of fatty acid mono- and diglycerides, of fatty acid sorbitan esters and alkyl (oligo)glucosides and fatty acid amidoalkyl betaines.

One group of surfactants that is to be used with preference is the anionic surfactants. Oral and dental hygiene and cleaning agents which are preferred according to the invention are characterized in that they contain 0.25 to 4% by weight, preferably 0.5 to 3.5% by weight, more preferably 0.75 to 3% by weight, even more preferably 1 to 2.5% by weight and in particular 1.6 to 2.2% by weight anionic surfactant(s).

Typical examples of anionic surfactants are soaps, alkylbenzene sulfonates, alkane sulfonates, olefin sulfonates, alkyl ether sulfonates, glycerol ether sulfonates, α-methyl ester sulfonates, sulfo fatty acids, alkyl sulfates, fatty alcohol ether sulfates, glycerol ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acyl amino acids, such as for example acyl lactylates, acyl tartrates, acyl glutamates and acyl aspartates, alkyl oligoglucoside sulfates, protein fatty acid condensates (in particular wheat-based plant products) and alkyl (ether) phosphates. If the anionic surfactants contain polyglycol ether chains, these may have a conventional homolog distribution, but preferably a narrowed homolog distribution.

Very particularly preferred agents according to the invention contain alkyl sulfate(s) as the anionic surfactant. Here, oral and dental hygiene and cleaning agents which are particularly preferred according to the invention are characterized in that they contain 0.25 to 4% by weight, preferably 0.5 to 3.5% by weight, more preferably 0.75 to 3% by weight, even more preferably 1 to 2.5% by weight and in particular 1.6 to 2.2% by weight sodium dodecyl sulfate.

With particular preference, the agents according to the invention contain amphoteric surfactant(s) in addition to or as an alternative to the anionic surfactants. Ampholytic surfactants and emulsifiers are understood to mean those surface-active compounds which contain, besides a C₈-C₂₄ alkyl or acyl group, at least one free amino group and at least one —COOH— or —SO₃H group and which are capable of forming internal salts. Examples of suitable ampholytic surfactants are N-alkyl glycines, N-alkylaminopropionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids having in each case approximately 8 to 24 carbon atoms in the alkyl group. Particularly preferred ampholytic surfactants are N-cocoalkyl aminopropionate, cocoacylaminoethyl aminopropionate and C₁₂-C₁₈ acyl sarcosine.

Particularly preferred oral and dental hygiene and cleaning agents according to the invention are characterized in that they contain 0.01 to 2% by weight, preferably 0.05 to 1.5% by weight, more preferably 0.1 to 1% by weight, even more preferably 0.12 to 0.7% by weight and in particular 0.15 to 0.6% by weight amphoteric surfactant(s).

Particularly preferred oral and dental hygiene and cleaning agents according to the invention are characterized in that they contain amphoteric surfactant(s) from the group consisting of
N-alkyl glycines,
N-alkylpropionic acids,
N-alkylaminobutyric acids,
N-alkyliminodipropionic acids,
N-hydroxyethyl-N-alkylamidopropyl glycines,
N-alkyl taurines,
N-alkyl sarcosines,
2-alkylaminopropionic acids having in each case approximately 8 to 24 carbon atoms in the alkyl group,
alkylaminoacetic acids having in each case approximately 8 to 24 carbon atoms in the alkyl group,
N-cocoalkyl aminopropionate,
cocoacylaminoethyl aminopropionate
C₁₂-C₁₈ acyl sarcosine,
N-alkyl-N,N-dimethyl ammonium glycinates, for example cocoalkyl dimethyl ammonium glycinate,
N-acylaminopropyl-N,N-dimethyl ammonium glycinates, for example cocoacyl aminopropyl dimethyl ammonium glycinate,
2-alkyl-3-carboxymethyl-3-hydroxyethyl imidazolines having in each case 8 to 18 carbon atoms in the alkyl or acyl group,
cocoacyl aminoethyl hydroxyethyl carboxymethyl glycinate,
the compounds known by the INCI name cocamidopropyl betaine,
the compounds known by the INCI name disodium cocoamphodiacetate.

Particularly preferred oral and dental hygiene and cleaning agents contain, as amphoteric surfactants, betaines of formula (Bet-I)

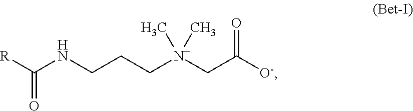

in which R represents a straight-chain or branched, saturated or mono- or polyunsaturated alkyl or alkenyl radical having 8 to 24 carbon atoms.

Under the INCI nomenclature, these surfactants are known as amidopropyl betaines, wherein the representatives derived from coco fatty acids are preferred and are known as cocoamidopropyl betaines. According to the invention, use is particularly preferably made of surfactants of formula (Bet-I) which are a mixture of the following representatives:
H₃C—(CH₂)₇—C(O)—NH—(CH₂)₃N⁺(CH₃)₂CH₂COO⁻
H₃C—(CH₂)₉—C(O)—NH—(CH₂)₃N⁺(CH₃)₂CH₂COO⁻
H₃C—(CH₂)₁₁—C(O)—NH—(CH₂)₃N⁺(CH₃)₂CH₂COO⁻
H₃C—(CH₂)₁₃—C(O)—NH—(CH₂)₃N⁺(CH₃)₂CH₂COO⁻
H₃C—(CH₂)₁₅—C(O)—NH—(CH₂)₃N⁺(CH₃)₂CH₂COO⁻
H₃C—(CH₂)₇—CH═CH—(CH₂)₇—C(O)—NH—(CH₂)₃N⁺(CH₃)₂CH₂COO⁻

Surfactants of formula (Bet-I) are particularly preferably used within relatively narrow quantity ranges. Here, preference is given to oral and dental hygiene and cleaning agents according to the invention which contain—relative to their weight—0.01 to 2% by weight, preferably 0.05 to 1.5% by weight, more preferably 0.1 to 1% by weight, even more preferably 0.12 to 0.7% by weight and in particular 0.15 to 0.6% by weight cocoamidopropyl betaines.

According to the invention, the weight ratio of polishing agent(s) to surfactant(s) is ≦6, that is to say the polishing agents are used at most in a 6-fold (weight) excess relative to the surfactants. The weight ratio relates to the weight ratio of all the substances from the aforementioned groups relative to one another, wherein only the active substances are taken into account. If an agent according to the invention includes for example 12% by weight polishing agent and 4% by weight of a 50% strength surfactant solution, the weight ratio is 12/2=6. If an agent according to the invention contains for example 12% by weight polishing agent and 4% by weight of a 50% strength surfactant solution and also 1% by weight pure sodium dodecyl sulfate, the weight ratio is 12/(2+1)=4.

In agents which are preferred according to the invention, the weight ratio lies within an even narrower range. Particularly preferred oral and dental hygiene and cleaning agents are characterized in that the weight ratio of polishing agent(s) to surfactant(s) lies in the range ≧5 to ≦15, preferably in the range ≧7.5 to ≦12.5, more preferably in the range ≧10 to ≦12 and in particular in the range ≧10.25 to ≦11.9.

The agents according to the invention, in particular the toothpastes, may also contain substances for increasing the mineralizing potential, for example calcium-containing substances such as, for example, calcium chloride, calcium acetate and dicalcium phosphate dihydrate. The concentration of the calcium-containing substance depends on the solubility of the substance and the interaction with other substances contained in the oral and dental hygiene agent.

Besides the aforementioned obligatory components, the dental hygiene agents according to the invention may contain further auxiliaries and additives which are known per se. One additive, which has been known as a toothpaste component for a long time, is particularly effective in the dental hygiene agents according to the invention: calcium glycerophosphate, the calcium salt of 1-glycerophosphoric acid or of 2-glycerophosphoric acid or of 3-glycerophosphoric acid, which is the enantiomer of 1-glycerophosphoric acid, or a mixture of said acids. In dental hygiene agents, the compound has a remineralizing effect since it supplies both calcium ions and phosphate ions. In the dental hygiene agents according to the invention, calcium glycerophosphate is used preferably in quantities of 0.01-1% by weight. In total, the dental cleaning agents according to the invention may contain customary auxiliaries and additives in quantities up to 10% by weight.

The organoleptic properties of the dental hygiene agents according to the invention can be improved for example by adding flavoring oils and sweeteners.

As flavoring oils, all the natural and synthetic flavorings customary for oral and dental hygiene agents may be used. Natural flavorings may be present both in the form of the natural essential oils isolated from drugs and in the form of the individual components isolated from said oils.

Suitable flavorings are, for example, peppermint oil, spearmint oil, eucalyptus oil, anise oil, fennel oil, caraway oil, menthyl acetate, cinnamic aldehyde, anethole, vanillin, thymol and mixtures of said components.

Suitable sweeteners are, for example, sodium saccharin, sodium cyclamate, sucrose, lactose, meltose, fructose.

Further customary auxiliaries and additives for toothpastes and mouthwashes or mouth rinsing solutions are
surface-active substances, preferably anionic, zwitterionic, amphoteric or nonionic surfactants or a combination of a plurality of different surfactants
solvents and solubilizers, for example lower monovalent or polyvalent alcohols or ethers, for example ethanol, 1,2-propylene glycol, diethylene glycol or butyl diglycol
pigments, such as for example titanium dioxide
colorants
buffer substances, for example primary, secondary or tertiary alkali phosphates or citric acid/sodium citrate
further wound-healing or anti-inflammatory substances, for example allantoin, urea, azulene, chamomile active substances, acetylsalicylic acid derivatives or rhodanide
further vitamins such as, for example, ascorbic acid, biotin, tocopherol or rutin
mineral salts such as, for example, manganese, zinc or magnesium salts.

It has been found that the performance of the oral and dental hygiene and cleaning agents according to the invention can be further increased if the agents contain salivation-promoting substances. In particular, the antibacterial effect and hence the anti-cavity effect and the effect against gingivitis and/or periodontitis are amplified as a result.

Salivation is understood to mean the production and release of saliva, moreover also in an unphysiologically increased quantity. Substances which encourage the flow of saliva and increase the quantity of saliva and/or release of saliva can stem from a wide range of substance classes.

One substance which is suitable for example according to the invention is pilocarpine, which may be contained in the oral and dental hygiene and cleaning agents according to the invention.

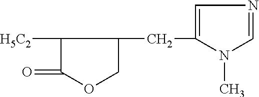

### Pilocarpine

Further salivation-promoting substances are in particular so-called pungent substances, that is to say substances which have a pungent taste and/or which give rise to a feeling of heat. Oral and dental hygiene and cleaning agents which are preferred according to the invention are characterized in that they contain, as salivation-promoting substances, at least one substance which has a pungent taste and/or which gives rise to a feeling of heat.

As the salivation-promoting ingredient, the products of this embodiment according to the invention contain a substance which has a pungent taste and/or which gives rise to a feeling of heat. These substances impart to the user a pungent, tingling, mouth-watering or heat-generating effect, that is to say they cause in sensory terms an impression of heat or a burning, or a tingling, bubbly, tickly or effervescent sensation, and thus promote the flow of saliva.

Products of this embodiment which are preferred according to the invention contain the substance(s) which has(have) a pungent taste and/or which give(s) rise to a feeling of heat in quantities of 0.00001 to 5% by weight, preferably 0.0005 to 2.5% by weight, more preferably 0.001 to 1% by weight, particularly preferably 0.005 to 0.75% by weight and in particular 0.01 to 0.5% by weight, in each case relative to the weight of the agent as a whole.

A range of substances may be used as the substance which has a pungent taste or which gives rise to a feeling of heat. Preference is given in particular to N-alkyl-substituted amides of unsaturated carboxylic acids, for example
2E,4E-decadienoic acid N-methylamide
2E,4E-decadienoic acid N-ethylamide
2E,4E-decadienoic acid N-n-propylamide
2E,4E-decadienoic acid N-isopropylamide
2E,4E-decadienoic acid N-n-butylamide
2E,4E-decadienoic acid N-(1-methylpropyl)amide
2E,4E-decadienoic acid N-isobutylamide
2E,4E-decadienoic acid N-tert-butylamide
2E,4Z-decadienoic acid N-methylamide
2E,4Z-decadienoic acid N-ethylamide
2E,4Z-decadienoic acid N-n-propylamide
2E,4Z-decadienoic acid N-isopropylamide
2E,4Z-decadienoic acid N-n-butylamide
2E,4Z-decadienoic acid N-(1-methylpropyl)amide
2E,4Z-decadienoic acid N-isobutylamide
2E,4Z-decadienoic acid N-tert-butylamide
2E,4E,8Z-decatrienoic acid N-methylamide
2E,4E,8Z-decatrienoic acid N-ethylamide
2E,4E,8Z-decatrienoic acid N-n-propylamide
2E,4E,8Z-decatrienoic acid N-isopropylamide
2E,4E,8Z-decatrienoic acid N-n-butylamide
2E,4E,8Z-decatrienoic acid N-(1-methylpropyl)amide
2E,4E,8Z-decatrienoic acid N-isobutylamide
2E,4E,8Z-decatrienoic acid N-tert-butylamide
2E,4Z,8Z-decatrienoic acid N-methylamide
2E,4Z,8Z-decatrienoic acid N-ethylamide
2E,4Z,8Z-decatrienoic acid N-n-propylamide
2E,4Z,8Z-decatrienoic acid N-isopropylamide
2E,4Z,8Z-decatrienoic acid N-n-butylamide
2E,4Z,8Z-decatrienoic acid N-(1-methylpropyl)amide
2E,4Z,8Z-decatrienoic acid N-isobutylamide
2E,4Z,8Z-decatrienoic acid N-tert-butylamide
2E,4E,8E-decatrienoic acid N-methylamide
2E,4E,8E-decatrienoic acid N-ethylamide
2E,4E,8E-decatrienoic acid N-n-propylamide
2E,4E,8E-decatrienoic acid N-isopropylamide
2E,4E,8E-decatrienoic acid N-n-butylamide
2E,4E,8E-decatrienoic acid N-(1-methylpropyl)amide
2E,4E,8E-decatrienoic acid N-isobutylamide
2E,4E,8E-decatrienoic acid N-tert-butylamide
2E,4Z,8E-decatrienoic acid N-methylamide
2E,4Z,8E-decatrienoic acid N-ethylamide
2E,4Z,8E-decatrienoic acid N-n-propylamide
2E,4Z,8E-decatrienoic acid N-isopropylamide
2E,4Z,8E-decatrienoic acid N-n-butylamide
2E,4Z,8E-decatrienoic acid N-(1-methylpropyl)amide
2E,4Z,8E-decatrienoic acid N-isobutylamide
2E,4Z,8E-decatrienoic acid N-tert-butylamide
2E,6Z,8E-decatrienoic acid N-methylamide
2E,6Z,8E-decatrienoic acid N-ethylamide
2E,6Z,8E-decatrienoic acid N-n-propylamide
2E,6Z,8E-decatrienoic acid N-isopropylamide
2E,6Z,8E-decatrienoic acid N-n-butylamide
2E,6Z,8E-decatrienoic acid N-(1-methylpropyl)amide
2E,6Z,8E-decatrienoic acid N-isobutylamide
2E,6Z,8E-decatrienoic acid N-tert-butylamide
2E,6E,8E-decatrienoic acid N-methylamide
2E,6E,8E-decatrienoic acid N-ethylamide
2E,6E,8E-decatrienoic acid N-n-propylamide
2E,6E,8E-decatrienoic acid N-isopropylamide
2E,6E,8E-decatrienoic acid N-n-butylamide
2E,6E,8E-decatrienoic acid N-(1-methylpropyl)amide
2E,6E,8E-decatrienoic acid N-isobutylamide
2E,6E,8E-decatrienoic acid N-tert-butylamide
2E,6Z,8Z-decatrienoic acid N-methylamide
2E,6Z,8Z-decatrienoic acid N-ethylamide
2E,6Z,8Z-decatrienoic acid N-n-propylamide
2E,6Z,8Z-decatrienoic acid N-isopropylamide
2E,6Z,8Z-decatrienoic acid N-n-butylamide
2E,6Z,8Z-decatrienoic acid N-(1-methylpropyl)amide
2E,6Z,8Z-decatrienoic acid N-isobutylamide
2E,6Z,8Z-decatrienoic acid N-tert-butylamide
2E,6E,8Z-decatrienoic acid N-methylamide
2E,6E,8Z-decatrienoic acid N-ethylamide
2E,6E,8Z-decatrienoic acid N-n-propylamide
2E,6E,8Z-decatrienoic acid N-isopropylamide
2E,6E,8Z-decatrienoic acid N-n-butylamide
2E,6E,8Z-decatrienoic acid N-(1-methylpropyl)amide
2E,6E,8Z-decatrienoic acid N-isobutylamide
2E,6E,8Z-decatrienoic acid N-tert-butylamide
2E,7Z,9E-undecatrienoic acid N-methylamide
2E,7Z,9E-undecatrienoic acid N-ethylamide
2E,7Z,9E-undecatrienoic acid N-n-propylamide
2E,7Z,9E-undecatrienoic acid N-isopropylamide
2E,7Z,9E-undecatrienoic acid N-n-butylamide
2E,7Z,9E-undecatrienoic acid N-(1-methylpropyl)amide
2E,7Z,9E-undecatrienoic acid N-isobutylamide
2E,7Z,9E-undecatrienoic acid N-tert-butylamide
2E,7E,9E-undecatrienoic acid N-methylamide
2E,7E,9E-undecatrienoic acid N-ethylamide
2E,7E,9E-undecatrienoic acid N-n-propylamide
2E,7E,9E-undecatrienoic acid N-isopropylamide
2E,7E,9E-undecatrienoic acid N-n-butylamide
2E,7E,9E-undecatrienoic acid N-(1-methylpropyl)amide
2E,7E,9E-undecatrienoic acid N-isobutylamide
2E,7E,9E-undecatrienoic acid N-tert-butylamide
2E,7Z,9Z-undecatrienoic acid N-methylamide
2E,7Z,9Z-undecatrienoic acid N-ethylamide
2E,7Z,9Z-undecatrienoic acid N-n-propylamide
2E,7Z,9Z-undecatrienoic acid N-isopropylamide
2E,7Z,9Z-undecatrienoic acid N-n-butylamide
2E,7Z,9Z-undecatrienoic acid N-(1-methylpropyl)amide
2E,7Z,9Z-undecatrienoic acid N-isobutylamide
2E,7Z,9Z-undecatrienoic acid N-tert-butylamide
2E,7Z,9E-undecatrienoic acid N-methylamide
2E,7Z,9E-undecatrienoic acid N-ethylamide
2E,7Z,9E-undecatrienoic acid N-n-propylamide
2E,7Z,9E-undecatrienoic acid N-isopropylamide
2E,7Z,9E-undecatrienoic acid N-n-butylamide
2E,7Z,9E-undecatrienoic acid N-(1-methylpropyl)amide
2E,7Z,9E-undecatrienoic acid N-isobutylamide
2E,7Z,9E-undecatrienoic acid N-tert-butylamide

Of course, other substitution patterns on the nitrogen atom are also possible and preferred, for example longer-chain n-alkyl radicals ( . . . -N-n-pentylamide, . . . -N-n-pentylamide, . . . -N-n-pentylamide, N-n-pentylamide, . . . -N-n-pentylamide, . . . -N-n-hexylamide, . . . -N-n-heptylamide, . . . -N-n-octylamide, . . . -N-n-nonylamide, . . . -N-n-decylamide, . . . -N-n-undecylamide, . . . -N-n-dodecylamide, . . . -N-n-tridecylamide, etc.) or disubstituted . . . -N,N-dialkylamides such as . . . -N,N-dimethylamide, . . . -N,N-diethylamide, . . . -N,N-di-n-propylamide, . . . -N,N-diisopropylamide, . . . -N,N-di-n-butylamide, . . . -N,N-di(1-methylpropyl)amide, . . . -N,N-diisobutylamide, . . . -N,N-di-tert-butylamide, . . . -N,N-methylethylamide, . . . -N,N-methyl-n-propylamide, . . . -N,N-methylisopropylamide, . . . -N,N-ethyl-n-propylamide, . . . -N,N-ethylisopropylamide, etc.

Among the aforementioned compounds, some are particularly preferred in the context of the present invention. These are listed below:

2E,6Z,8E-decatrienoic acid N-isobutylamide (N-isobutyl-2E,6Z,8E-decatrienamide, also called spilanthol or affinine):

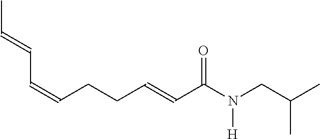 [SMILES: C/C=C/C=C\CC/C=C/C(=O)NCC(C)C]

2E,4E,8Z-decatrienoic acid N-isobutylamide (N-isobutyl-2E,4E,8Z-decatrienamide, also called isoaffinine):

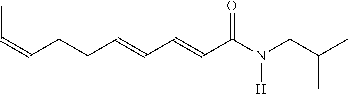 [SMILES: C/C=C\CC/C=C/C=C/C(=O)NCC(C)C]

2E,7Z,9E-undecatrienoic acid N-isobutylamide (N-isobutyl-2E,7Z,9E-undecatrienamide):

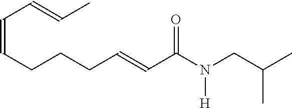 [SMILES: C/C=C/C=C\CCC/C=C/C(=O)NCC(C)C]

2E,4Z-decadienoic acid N-isobutylamide (cis-pellitorine):

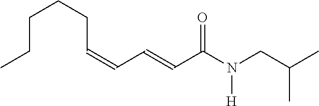 [SMILES: CCCCC/C=C\C=C\C(=O)NCC(C)C]

2E,4E-decadienoic acid N-isobutylamide (trans-pellitorine):

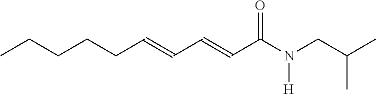 [SMILES: CCCCC/C=C/C=C/C(=O)NCC(C)C]

ferulic acid amides, for example
ferulic acid N-vanillylamide:

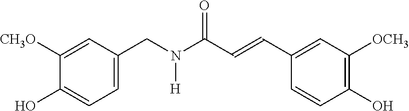

N-[2-(4-hydroxy-3-methoxyphenyl)ethyl]-3-(4-hydroxy-3-methoxyphenyl)-(2E)-propenoic acid amide (trans-feruloylmethoxytyramine):

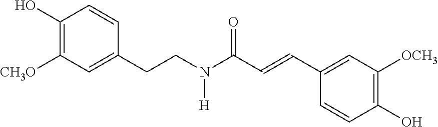

N-[2-(4-hydroxy-3-methoxyphenyl)ethyl]-3-(4-hydroxy-3-methoxyphenyl)-(2Z)-propenoic acid amide (cis-feruloylmethoxytyramine):

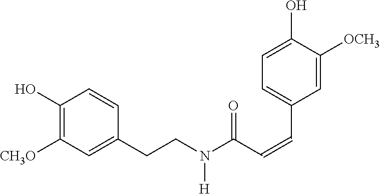

N-[2-(4-hydroxy-3-methoxyphenyl)ethyl]-3-(4-hydroxy-3-methoxyphenyl)-propanoic acid amide (dihydroferuloylmethoxytyramine):

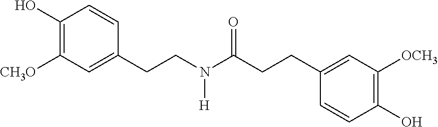

N-[2-(3,4-dihydroxyphenyl)ethyl]-3-(4-hydroxy-3-methoxyphenyl)-(2E)-propenoic acid amide (trans-feruloyldopamine):

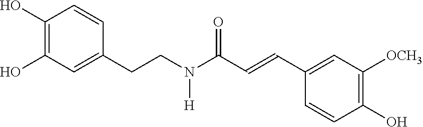 [SMILES: COc1cc(/C=C/C(=O)NCCc2ccc(O)c(O)c2)ccc1O]

N-[2-(3,4-dihydroxyphenyl)ethyl]-3-(4-hydroxy-3-methoxyphenyl)-(2Z)-propenoic acid amide (cis-feruloyldopamine):

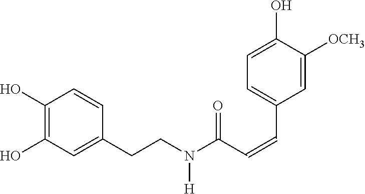 [SMILES: COc1cc(/C=C\C(=O)NCCc2ccc(O)c(O)c2)ccc1O]

N-[2-(4-hydroxyphenyl)ethyl]-3-(3,4-dihydroxyphenyl)-(2E)-propenoic acid amide (trans-caffeoyltyramine):

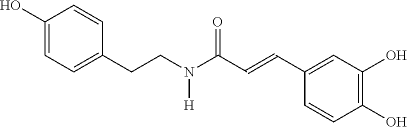 [SMILES: O=C(/C=C/c1ccc(O)c(O)c1)NCCc1ccc(O)cc1]

N-[2-(4-hydroxyphenyl)ethyl]-3-(3,4-dihydroxyphenyl)-(2Z)-propenoic acid amide (cis-caffeoyltyramine):

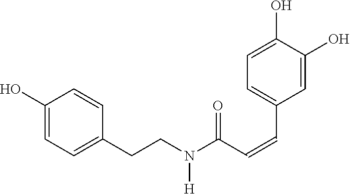 [SMILES: O=C(/C=C\c1ccc(O)c(O)c1)NCCc1ccc(O)cc1]

N-[2-(3,4-dimethoxyphenyl)ethyl]-3-(3,4-dimethoxyphenyl)-(2E)-propenoic acid amide (trans-rubenamine):

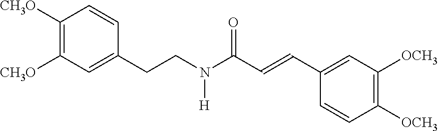

N-[2-(3,4-dimethoxyphenyl)ethyl]-3-(3,4-dimethoxyphenyl)-(2Z)-propenoic acid amide (cis-rubenamine):

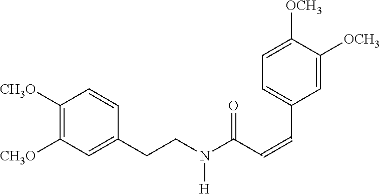

Further pungent substances which can be used with particular preference in the context of the present invention are, for example, extracts from natural plants. Plant extracts with a pungent taste may be all physiologically harmless plant extracts which give a pungent or hot sensory impression. Preferred plant extracts with a pungent taste are, for example, pepper extract (Piper ssp., in particular Piper nigrum), water pepper extract (Polygonum ssp., in particular Polygonum hydropiper), extracts of Allium ssp. (in particular onion and garlic extracts), extracts of radish (Raphanus ssp.), horseradish extracts (Cochlearia armoracia), extracts of black (Brassica nigra), wild or yellow mustard (Sinapis ssp., in particular Sinapis arvensis and Sinapis alba), pellitory root extracts (Anacyclus ssp., in particular Anacyclus pyrethrum L.), Echinacea extracts (Echinaceae ssp.), extracts of Szechuan pepper (Zanthoxylum ssp., in particular Zanthoxylum piperitum), Spilanthes extract (Spilanthes ssp., in particular Spilanthes acmella), chili extract (Capsicum ssp., in particular Capsicum frutescens), extract of grains of paradise (Aframomum ssp., in particular Aframomum melegueta [Rose] K. Schum.), ginger extract (Zingiber ssp., in particular Zingiber officinale) and galangal extract (Kaempferia galanga or Alpinia galanga).

One particularly suitable substance is gingerol stemming from ginger extract. N-Ethyl-p-menthane-3-carboxamide (N-ethyl-5-methyl-2-isopropylcyclohexane carboxamide) can also be used.

Other substances which have a pungent taste or which give rise to a feeling of heat may be, for example, capsaicin, dihydrocapsaicin, gingerol, paradol, shogaol, piperine, carboxylic acid N-vanillylamides, in particular nonanoic acid N-vanillylamide, 2-alkenoic acid amides, in particular 2-nonenoic acid N-isobutylamide, 2-nonenoic acid N-4-hydroxy-3-methoxyphenylamide, alkyl ethers of 4-hydroxy-3-methoxybenzyl alcohol, in particular 4-hydroxy-3-methoxybenzyl n-butyl ether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylenedioxybenzyl alcohol, nicotinaldehyde, methyl nicotinate, propyl nicotinate, 2-butoxyethyl nicotinate, benzyl nicotinate, 1-acetoxychavicol, polygodial or isodrimeninol.

Preferred remineralizing products according to the invention are characterized in that they contain at least one pungent substance from the group consisting of N-alkyl-substituted amides of unsaturated carboxylic acids, preferably
a. 2E,6Z,8E-decatrienoic acid N-isobutylamide (spilanthol) and/or
b. 2E,4E,8Z-decatrienoic acid N-isobutylamide and/or
c. 2E,7Z,9E-undecatrienoic acid N-isobutylamide and/or
d. 2E,4Z-decadienoic acid N-isobutylamide (cis-pellitorine) and/or
e. 2E,4E-decadienoic acid N-isobutylamide (trans-pellitorine) and/or
f. ferulic acid N-vanillylamide and/or
g. N-[2-(4-hydroxy-3-methoxyphenyl)ethyl]-3-(4-hydroxy-3-methoxyphenyl)-(2E)-propenoic acid amide (trans-feruloylmethoxytyramine) and/or
h. N-[2-(4-hydroxy-3-methoxyphenyl)ethyl]-3-(4-hydroxy-3-methoxyphenyl)-(2Z)-propenoic acid amide (cis-feruloylmethoxytyramine) and/or
i. N-[2-(4-hydroxy-3-methoxyphenyl)ethyl]-3-(4-hydroxy-3-methoxyphenyl)-propanoic acid amide (dihydroferuloylmethoxytyramine) and/or
j. N-[2-(3,4-dihydroxyphenypethyl]-3-(4-hydroxy-3-methoxyphenyl)-(2E)-propenoic acid amide (trans-feruloyldopamine) and/or
k. N-[2-(3,4-dihydroxyphenypethyl]-3-(4-hydroxy-3-methoxyphenyl)-(2Z)-propenoic acid amide (cis-feruloyldopamine) and/or
l. N-[2-(4-hydroxyphenyl)ethyl]-3-(3,4-dihydroxyphenyl)-(2E)-propenoic acid amide (trans-caffeoyltyramine) and/or
m. N-[2-(4-hydroxyphenyl)ethyl]-3-(3,4-dihydroxyphenyl)-(2Z)-propenoic acid amide (cis-caffeoyltyramine) and/or
n. N-[2-(3,4-dimethoxyphenypethyl]-3-(3,4-dimethoxyphenyl)-(2E)-propenoic acid amide (trans-rubenamine) and/or
o. N-[2-(3,4-dimethoxyphenypethyl]-3-(3,4-dimethoxyphenyl)-(2Z)-propenoic acid amide (cis-rubenamine).

In addition to the aforementioned pungent substances or in place thereof, further substances which have a pungent taste and/or which give rise to a feeling of heat may also be incorporated in the products according to the invention.

Alkyl-substituted dioxanes of formula

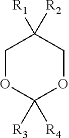

in which R1 and R2 independently of one another are selected from —H, —CH₃, —CH₂CH₃ and R3 and R4 independently of one another are selected from —H, —CH₃, —CH₂CH₃, have proven to be particularly suitable in the context of the present invention.

Phenyl esters of formula

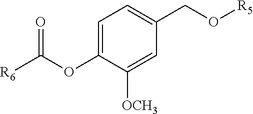

in which R5 represents —CH₃ or a straight-chain or branched alkyl or alkenyl radical having 2 to 8 carbon atoms and R6 represents CH₃ or a straight-chain or branched alkyl or alkenyl radical having 2 to 8 carbon atoms or an alkoxy group having 1 to 3 carbon atoms, have also proven to be particularly suitable in the context of the present invention.

Carvone acetals of formula

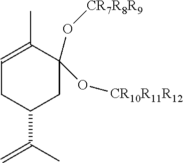

in which R7 to R12 independently of one another are selected from —H, —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃ or R9 and R10 together are a chemical bond or a group —(CR13R14)ₓ, in which x represents the values 1 or 2 and R13 and R14 independently of one another are selected from —H, —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃, have also proven to be particularly suitable in the context of the present invention.

Agents according to the invention can be formulated as toothpastes or tooth creams.

Another subject matter of the present invention is the use of agents according to the invention for cleaning teeth by means of manual or electric toothbrushes.

Another subject matter of the present invention is a method for cleaning teeth, characterized in that an agent according to the invention is applied to the brush head of an electric toothbrush and the teeth are cleaned using the electric toothbrush.

Another subject matter of the present invention is a method for cleaning teeth, characterized by the steps
a. providing a toothbrush, the brush head of which can be set in motion electrically;
b. applying 0.5 to 5 g of an agent according to the invention to the brush head,
c. cleaning the teeth with the agent according to the invention for 30 to 300 seconds using the brush head that has been set in motion electrically.

With regard to preferred embodiments of the use according to the invention and of the methods according to the invention, what has been stated above in relation to the agents according to the invention applies mutatis mutandis.

The compositions according to the invention can also be formulated as mouth rinsing solutions or mouthwashes. Another subject matter of the present invention is a method for preventing and treating cavities and/or for combating halitosis and/or for treating gingivitis or periodontitis, in which a preparation according to the invention in the form of a mouth rinsing solution is introduced into the oral cavity and is left therein for a duration of at least 10 seconds, preferably at least 20 seconds and in particular at least 45 seconds.

By using the polylactic acid particles, the taste and the feeling in the mouth can be considerably improved. Another subject matter of the present invention is therefore the use of polylactic acid particles to improve the taste of oral and dental hygiene and cleaning agents which contain sorbitol and/or glycerol.

### Examples

Mouth rinsing solutions (all data given in % by weight):

| | M1 | M2 | M3 | M4 | M5 | M6 | M7 | M8 |
|---|---|---|---|---|---|---|---|---|
| Sorbitol | 0 | 3 | 6 | 9 | 12 | 15 | 2 | 5 |
| Glycerol | 10 | 8 | 6 | 4 | 1 | 0 | 10 | 5 |
| Polylactic acid | 0.1 | 1.0 | 2.5 | 5.0 | 7.5 | 10.0 | 12.5 | 15.0 |
| particles (1) | | | | | | | | |
| Sodium fluoride | 0.05 | 0.05 | 0.05 | 0.05 | 0.05 | 0.045 | 0.045 | 0.045 |
| Sodium saccharin | 0.03 | 0.03 | 0.03 | 0.03 | 0.03 | 0.03 | 0.03 | 0.03 |
| PEG-60 hydrogenated | 0.86 | 0.86 | 0.86 | 0.86 | 0.86 | 0.86 | 0.86 | 0.86 |
| castor oil | | | | | | | | |
| Flavoring | 0.1 | 0.1 | 0.1 | 0.1 | 0.1 | 0.1 | 0.1 | 0.1 |
| Water, demineralized | ad | ad | ad | ad | ad | ad | ad | ad |
| | 100 | 100 | 100 | 100 | 100 | 100 | 100 | 100 |
| | M9 | M10 | M11 | M12 | M13 | M14 | M15 | M16 |
| Sorbitol | 5 | 7.5 | 10 | 12.5 | 15 | 10 | 7.5 | 5 |
| Polylactic acid | 0.1 | 1.0 | 2.5 | 5.0 | 7.5 | 10.0 | 12.5 | 15.0 |
| particles (1) | | | | | | | | |
| Sodium fluoride | 0.05 | 0.05 | 0.05 | 0.05 | 0.05 | 0.045 | 0.045 | 0.045 |
| Sodium saccharin | 0.03 | 0.03 | 0.03 | 0.03 | 0.03 | 0.03 | 0.03 | 0.03 |
| PEG-60 hydrogenated | 0.86 | 0.86 | 0.86 | 0.86 | 0.86 | 0.86 | 0.86 | 0.86 |
| castor oil | | | | | | | | |
| Flavoring | 0.1 | 0.1 | 0.1 | 0.1 | 0.1 | 0.1 | 0.1 | 0.1 |
| Water, demineralized | ad | ad | ad | ad | ad | ad | ad | ad |
| | 100 | 100 | 100 | 100 | 100 | 100 | 100 | 100 |
| | M17 | M18 | M19 | M20 | M21 | M22 | M23 | M24 |
| Glycerol | 40 | 30 | 20 | 10 | 7.5 | 5 | 12.5 | 17 |
| Polylactic acid | 0.1 | 1.0 | 2.5 | 5.0 | 7.5 | 10.0 | 12.5 | 15.0 |
| particles (1) | | | | | | | | |
| Sodium fluoride | 0.05 | 0.05 | 0.05 | 0.05 | 0.05 | 0.045 | 0.045 | 0.045 |
| Sodium saccharin | 0.03 | 0.03 | 0.03 | 0.03 | 0.03 | 0.03 | 0.03 | 0.03 |
| PEG-60 hydrogenated | 0.86 | 0.86 | 0.86 | 0.86 | 0.86 | 0.86 | 0.86 | 0.86 |
| castor oil | | | | | | | | |
| Flavoring | 0.1 | 0.1 | 0.1 | 0.1 | 0.1 | 0.1 | 0.1 | 0.1 |
| Water, demineralized | ad | ad | ad | ad | ad | ad | ad | ad |
| | 100 | 100 | 100 | 100 | 100 | 100 | 100 | 100 |

(1): Ecoscrub ® 50 PC, maximum particle size 297 μm

Toothpastes of the following composition (all data given in % by weight) were produced:

| | E1 | E2 | E3 | E4 | E5 |
|---|---|---|---|---|---|
| Sorbitol | 10 | 11 | 12 | 13 | 13 |
| Glycerol | 20 | 21 | 22 | 22.5 | 22.5 |
| Ethanol (96%) | — | — | — | — | 2.0 |
| Precipitated silica: | 10.0 | 10.0 | 10.0 | 10.0 | 12.0 |
| Sident 8 | | | | | |
| Precipitated silica: | 8.5 | 8.5 | 8.5 | 8.5 | — |
| Sident 22S | | | | | |
| Poliertonerde P10 | — | 1.0 | — | — | — |
| feinst (polishing | | | | | |
| alumina) | | | | | |
| Disodium phosphate, | 0.1 | 0.1 | 0.1 | 0.1 | 0.1 |
| anhydrous | | | | | |
| Trisodium phosphate, | 0.2 | 0.2 | 0.2 | 0.2 | — |
| anhydrous | | | | | |
| Sodium monofluoro- | — | — | 1.0 | 1.0 | 1.0 |
| phosphate Na2PO3F | | | | | |
| Sodium fluoride | 0.32 | 0.32 | — | — | — |
| Polyethylene glycol | | | 1.5 | | |
| (MW: 1500) | | | | | |
| Titanium dioxide | 0.5 | 0.5 | 0.5 | 0.5 | — |
| Sodium lauryl sulfate | 1.5 | 1.5 | 1.5 | 1.5 | 1.5 |
| Sodium saccharin | 0.1 | 0.1 | 0.1 | 0.1 | 0.1 |
| Xanthan | 0.4 | 0.4 | 0.4 | 0.4 | 0.4 |
| Flavoring | 1.0 | 1.0 | 1.0 | 1.0 | 1.0 |
| Polylactic acid | 0.1 | 0.5 | 1.0 | 1.5 | 2.5 |
| particles (1) | | | | | |
| Water, demineralized | ad 100 | ad 100 | ad 100 | ad 100 | ad 100 |

(1): Ecoscrub ® 50 PC, maximum particle size 297 μm

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An oral and dental hygiene and cleaning agent, comprising, relative to its weight,
a) 0.001 to 25% by weight polylactic acid particles, and
b) 0 to 15% by weight sorbitol,
c) 0 to 30% by weight glycerol,
with the proviso that the total amount of ingredient(s) from groups b) and c) is 5 to 40% by weight.

2. The oral and dental hygiene and cleaning agent according to claim 1, wherein the agent comprises 0.002-20% by weight polylactic acid particles.

3. The oral and dental hygiene and cleaning agent according to claim 1, wherein the agent comprises 0.005-12.5% by weight polylactic acid particles.

4. The oral and dental hygiene and cleaning agent according to claim 1, wherein the polylactic acid particles have particle sizes of 1 to 1000 μm.

5. The oral and dental hygiene and cleaning agent according to claim 1, wherein the polylactic acid particles have particle sizes of 2 to 750 μm.

6. The oral and dental hygiene and cleaning agent according to claim 1, wherein the polylactic acid particles have mean ECD values of 10 to 1000 μm.

7. The oral and dental hygiene and cleaning agent according to claim 1, wherein the polylactic acid particles have mean ECD values of 100 to 350 μm.

8. The oral and dental hygiene and cleaning agent according to claim 1, wherein the polylactic acid particles have a mean solidity of 0.4 to 0.9.

9. The oral and dental hygiene and cleaning agent according to claim 1, wherein the polylactic acid particles have a mean solidity of 0.55 to 0.65.

10. The oral and dental hygiene and cleaning agent according to claim 1, wherein the polylactic acid particles have a mean circularity C of 0.1 to 0.6.

11. The oral and dental hygiene and cleaning agent according to claim 1, wherein the polylactic acid particles have a mean circularity C of 0.2 to 0.35.

12. The oral and dental hygiene and cleaning agent according to claim 1, further comprising 40 to 84% by weight water.

13. The oral and dental hygiene and cleaning agent according to claim 1, wherein the agent includes 1 to 14.5% by weight sorbitol.

14. The oral and dental hygiene and cleaning agent according to claim 1, wherein the agent includes 5 to 28% by weight glycerol.

15. The oral and dental hygiene and cleaning agent according to claim 1, wherein the total amount of sorbitol b) and glycerol c) is 20 to 35% by weight.

16. The oral and dental hygiene and cleaning agent according to claim 1, further comprising 1225 to 1575 ppm fluoride.

17. The oral and dental hygiene and cleaning agent according to claim 1, further comprising 5 to 25% by weight of a polishing agent selected from the group consisting of silicas, aluminum hydroxide, aluminum oxide, dicalcium phosphate dihydrate (CaHPO₄.2H₂O) or mixtures thereof.

18. The oral and dental hygiene and cleaning agent according to claim 1, further comprising 0.25 to 4% by weight anionic surfactant.
